(19) United States
(12) Patent Application Publication
(10) Pub. No.: US 2022/0051796 A1
(43) Pub. Date: Feb. 17, 2022

(54) METHOD AND DATA PROCESSING APPARATUS FOR GENERATING REAL-TIME ALERTS ABOUT A PATIENT

(71) Applicant: Oxford University Innovation Limited, Oxford Oxfordshire (GB)

(72) Inventors: Tingting ZHU, Oxford Oxfordshire (GB); Farah SHAMOUT, Oxford Oxfordshire (GB); David CLIFTON, Oxford Oxfordshire (GB); Peter WATKINSON, Oxford Oxfordshire (GB)

(21) Appl. No.: 17/299,155

(22) PCT Filed: Dec. 5, 2019
(86) PCT No.: PCT/GB2019/053437

§ 371 Date: Jun. 2, 2021

(30) Foreign Application Priority Data
Dec. 7, 2018 (GB) 1820004.8

(51) Int. Cl. G16H 50/20; G16H 50/70; G06N 3/04

(52) U.S. Cl. CPC G16H 50/20; G06N 3/0454; G16H 50/70

(57) ABSTRACT

This disclosure relates to methods and apparatus for generating real-time alerts about a patient. In one arrangement, vital sign data representing vital sign information obtained from the patient at one or more input times within an assessment time window is received. A Gaussian process model of at least a portion of the vital sign information is used to generate a time series of synthetic vital sign data based on the received vital sign data, the synthetic vital sign data comprising at least a posterior mean for each of one or more components of the vital sign information at each of a plurality of regularly spaced time points in the assessment time window. The generated synthetic vital sign data is used as input to a trained recurrent neural network to generate an early warning score, the early warning score representing a probability of an adverse event occurring during a prediction time window of predetermined length after the assessment time window. An alert is generating about the patient dependent on the generated early warning score.

The invention relates to generating real-time alerts about a patient using an Early Warning Score (EWS) generated using vital sign information.

Increased access to Electronic Health Records (EHR) has motivated the development of data-driven systems that detect physiological derangement and secure timely response. Commonly predicted adverse events such as mortality, unplanned ICU admission and cardiac arrest, have been extensively investigated by EWS systems, such as the National Early Warning Score (NEWS) that is currently recommended by the Royal College of Physicians in the UK. Typically, EWS systems assign a real-time alerting score to a set of vital sign measurements based on predetermined normality thresholds to indicate the patient's degree of illness.

However, physiological data recorded in EHRs are often sparse, noisy and incomplete, especially when collected in non-critical care wards. Missingness is often dealt with through complete-case analysis, population mean imputation, or carrying the most recent value forward. Such practices may impose bias and error and do not account for the uncertainty of the imputed data.

It is an object of the invention to at least partly address one or more of the issues described above.

According to an aspect, there is provided a computer-implemented method of generating real-time alerts about a patient, comprising: receiving vital sign data representing vital sign information obtained from the patient at one or more input times within an assessment time window; using a Gaussian process model of at least a portion of the vital sign information to generate a time series of synthetic vital sign data based on the received vital sign data, the synthetic vital sign data comprising at least a posterior mean for each of one or more components of the vital sign information at each of a plurality of regularly spaced time points in the assessment time window; using the generated synthetic vital sign data as input to a trained recurrent neural network to generate an early warning score, the early warning score representing a probability of an adverse event occurring during a prediction time window of predetermined length after the assessment time window; and generating an alert about the patient dependent on the generated early warning score.

Thus, a method is provided in which Gaussian process regression is used to generate synthetic vital sign data at regularly spaced intervals, which is provided as input to a recurrent neural network (RNN). This combination of processing architectures can be implemented efficiently using relatively modest computational resource and is demonstrated to achieve a high level of performance in generating EWSs. The architecture allows long term dependencies to be summarized efficiently. The Gaussian process regression allows computationally efficient modelling, where population based priors can be used to set up the Gaussian process model and the architecture as a whole achieves personalized modelling efficiently.

In an embodiment, the recurrent neural network comprises an attention mechanism.

The inventors have demonstrated that the introduction of an attention mechanism to the recurrent neural network provides a significant increase in performance. Furthermore, the attention mechanism provides the basis for improved interpretability by identifying which time points and/or which components of vital sign information are most relevant to the generated EWS.

In an embodiment, the recurrent neural network comprises a bidirectional Long Short Term Memory network.

The inventors have demonstrated that particularly high performance is achieved where the recurrent neural network is implemented as a bidirectional Long Short Term Memory (LSTM) network.

In an embodiment, the synthetic vital sign data comprises a posterior variance corresponding to each posterior mean; each posterior mean corresponding to each time point is used as input to a first recurrent neural network; each posterior variance corresponding to each time point is used as input to a second recurrent neural network; and the early warning score is generated via processing of outputs from both the first recurrent neural network and the second recurrent neural network. Furthermore, the first recurrent neural network interacts with an attention mechanism; the attention mechanism computes a respective attention weight to apply to a hidden state of the first recurrent neural network corresponding to each time point in the assessment time window; and the early warning score is generated via processing of a combination of a weighted sum of the hidden states of the first recurrent neural network weighted by the computed attention weights and an output from the second recurrent neural network.

The inventors have demonstrated that incorporating posterior variances further improves performance.

In an embodiment, the first recurrent neural network interacts with a first attention mechanism; the second recurrent neural network interacts with a second attention mechanism; the first attention mechanism computes a respective attention weight to apply to a hidden state of the first recurrent neural network corresponding to each time point in the assessment time window; the second attention mechanism computes a respective attention weight to apply to a hidden state of the second recurrent neural network corresponding to each time point in the assessment time window; and the early warning score is generated via processing of a combination of a weighted sum of the hidden states of the first recurrent neural network weighted by the computed attention weights of the first attention mechanism and a weighted sum of the hidden states of the second recurrent neural network weighted by the computed attention weights of the second attention mechanism.

The inventors have demonstrated that incorporating posterior means and variances via separate attention mechanisms further improves performance.

In an embodiment, the method further comprises receiving laboratory test data representing information obtained from one or more laboratory tests performed on the patient; receiving a diagnosis code representing a diagnosis of the patient made at a time of admission of the patient to a medical facility; using a trained model of a relationship between laboratory test data and probabilities of an adverse event occurring during the prediction time window to generate an early warning score based on the laboratory test data; using a trained model of a relationship between diagnosis codes and probabilities of an adverse event occurring during the prediction time window to generate an early warning score based on the diagnosis code; and obtaining a composite early warning score using a combination of at least the early warning score generated using the trained recurrent neural network, the early warning score based on the laboratory test data, and the early warning score based on the diagnosis code, wherein the alert is generated using the composite early warning score.

The inventors have demonstrated that the generation of alerts can be improved by such fusing of early warning scores obtained based on vital sign data, laboratory test data, and diagnosis codes.

In an embodiment, the model of the relationship between laboratory test data and probabilities of an adverse event includes a decay term to model an effect of delay between obtaining of the laboratory test data and a time at which the composite early warning score is to be obtained. The inventors have found that modelling the effect of delay in this way further improves the generation of alerts.

Embodiments of the invention will now be described, by way of example only, with reference to the accompanying drawings in which corresponding reference symbols indicate corresponding parts, and in which:

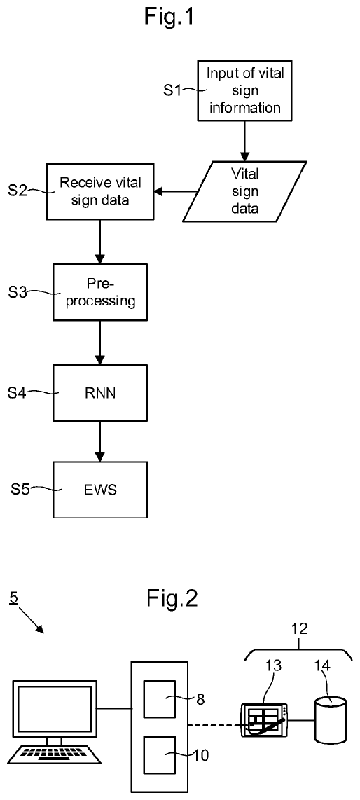

FIG. 1 is a flow chart schematically depicting a method of generating early warning scores for generating alerts about a patient in real time;

FIG. 2 depicts a data processing apparatus configured to receive vital sign data from a sensor system;

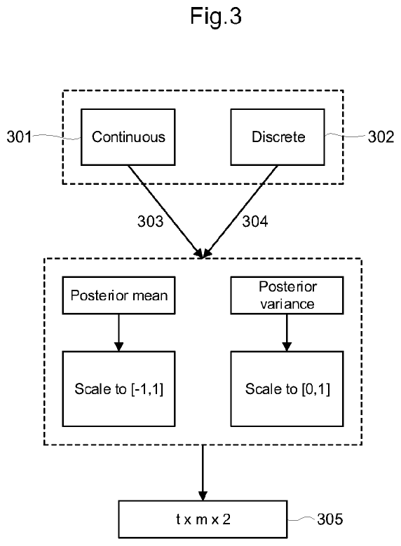

FIG. 3 depicts example pre-processing steps for continuous and discrete time series variables to obtain a feature space for input to a recurrent neural network;

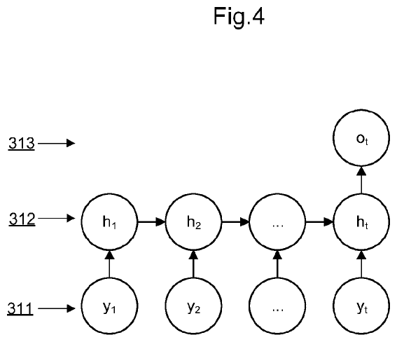

FIG. 4 depicts a simple LSTM classification model architecture;

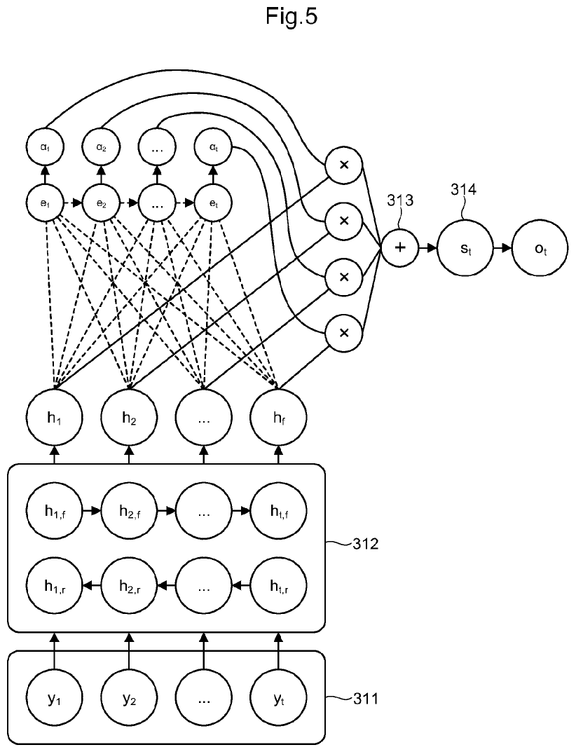

FIG. 5 depicts an LSTM-ATT classification model architecture which learns from and applies the attention weights to the mean input only;

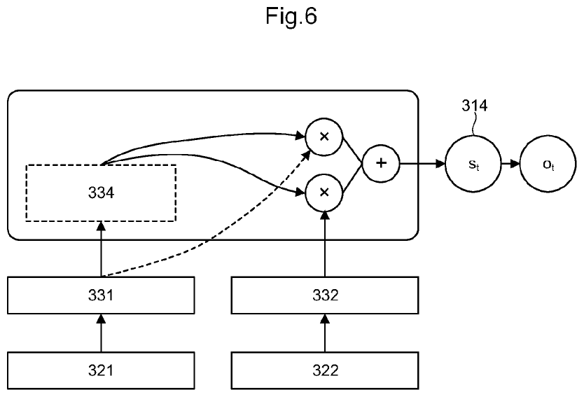

FIG. 6 depicts a UA-LSTM-ATT-1 classification model architecture which learns the attention weights from the mean input and applies it to the hidden states of the mean and variance inputs;

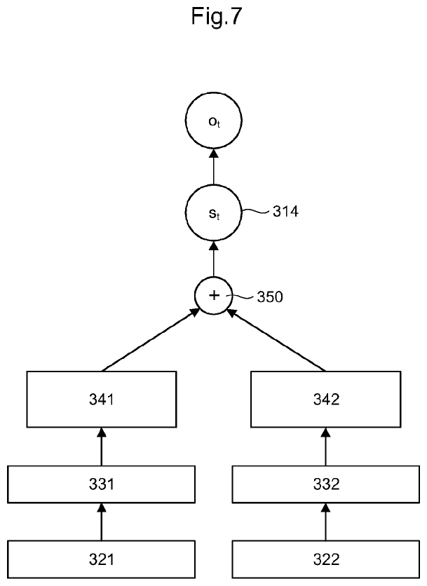

FIG. 7 depicts a UA-LSTM-ATT-2 classification model architecture which learns the attention weights and context vectors from the mean and variance inputs independently;

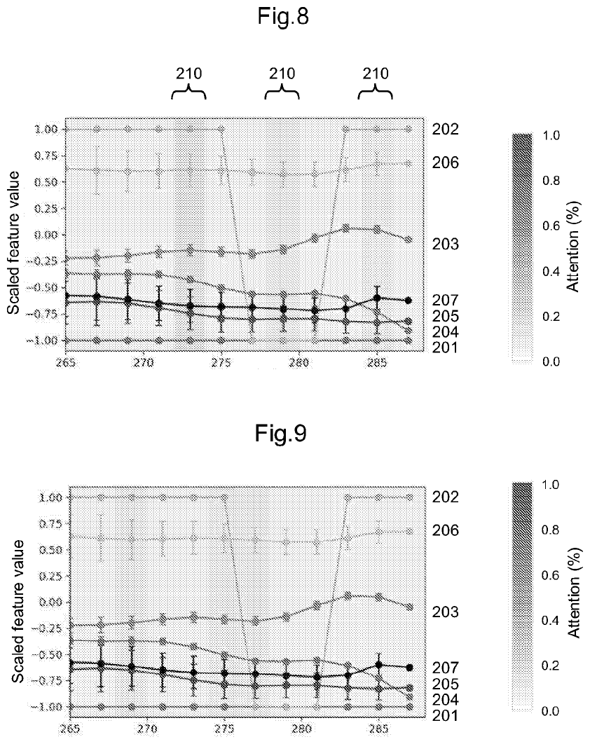

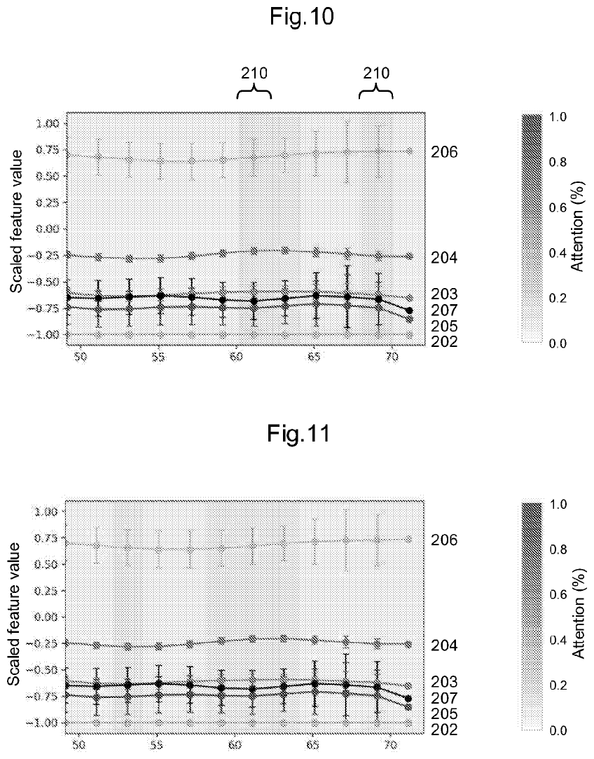

FIGS. 8-11 compare attention weightings of an attention layer at different time points by the LSTM-ATT model (FIGS. 9 and 11) and the UA-LSTM-ATT (FIGS. 8 and 10) for two test patients: one deteriorating patient (FIGS. 8 and 9) and one non-deteriorating patient (FIGS. 10 and 11); the mean and variance of vital signs features obtained after data pre-processing are also visualized;

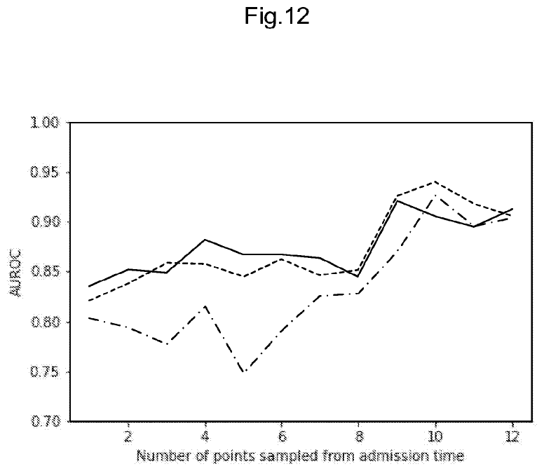

FIG. 12 is a graph providing a performance comparison of different classification models in terms of Area under Receiving Operating Characteristic (AUROC) Curve on test sequences of varying length size, ranging between 1 and 12 points within a 24 hour window of observations and excluding pre-padded data points;

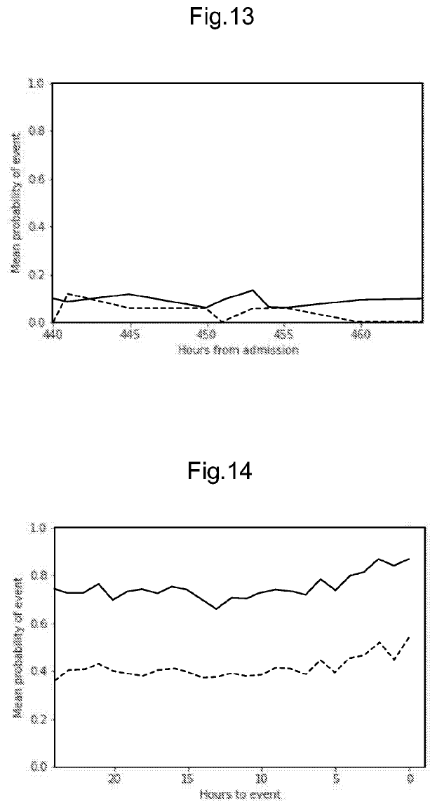

FIGS. 13-14 are graphs comparing mean alerting probability of NEWS and the UA-LSTM-ATT-2 classification model for non-deteriorating patients in a sample hospitalization window (FIG. 13) and deteriorating patients in the 24 hours window prior to an event (FIG. 14);

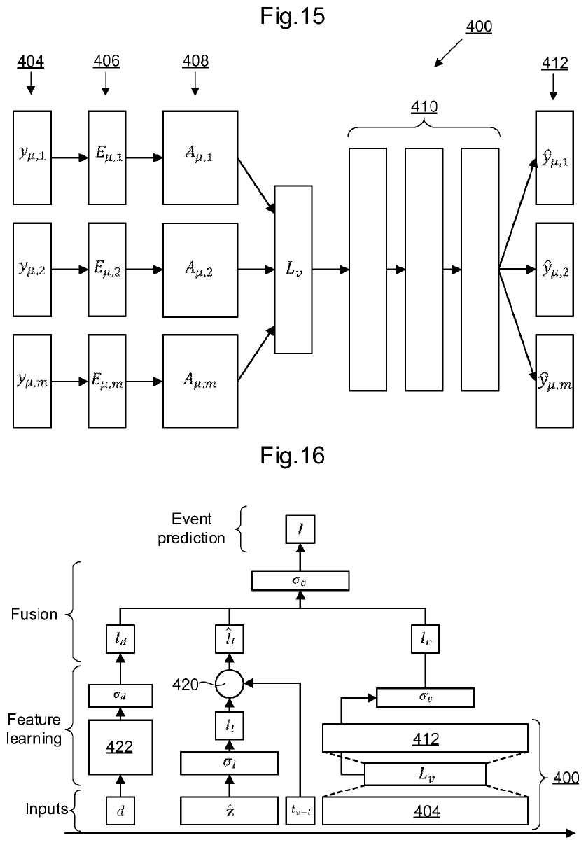

FIG. 15 schematically depicts an autoencoder-based architecture for unsupervised feature learning from vital sign data;

FIG. 16 schematically depicts a model configured to learn from vital sign data, laboratory test data and diagnosis codes;

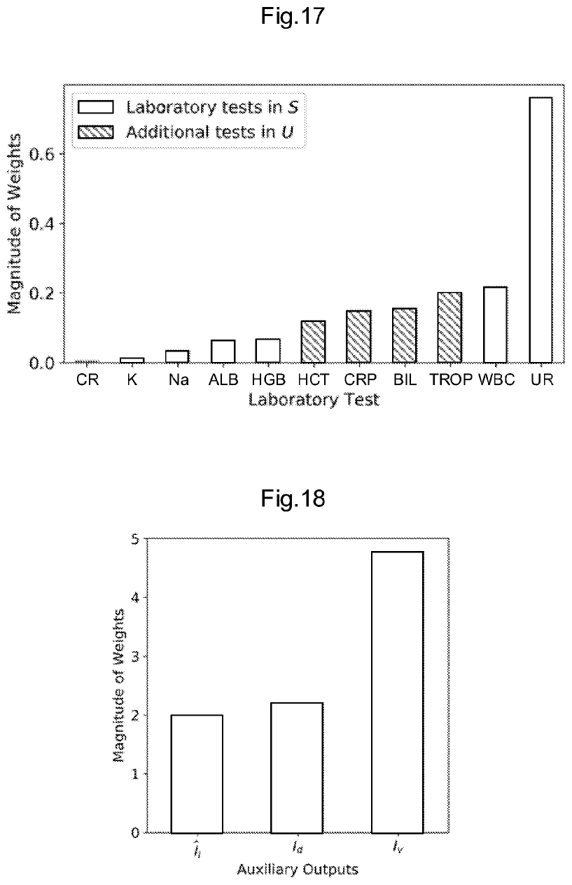

FIG. 17 is a graph depicting the absolute value of weights assigned to laboratory test data variables;

FIG. 18 is a graph providing visualisation of the magnitude of coefficients assigned to auxiliary outputs during generation of a composite early warning score; and

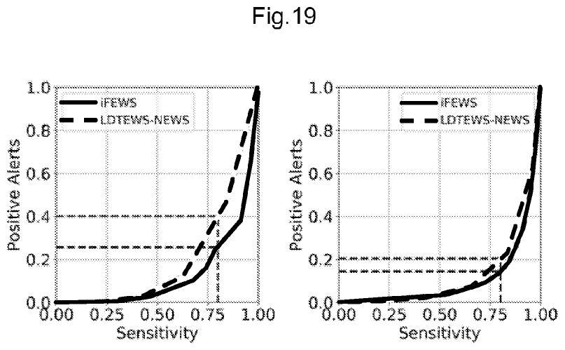

FIG. 19 depict efficiency curves plotting sensitivity (horizontal axis) against the percentage of observations (vertical axis) with an early warning score greater than or equal to a decision threshold (left graph was derived for 16-45 years old patient group; right graph was derived for >45 years old patient group).

Methods of the present disclosure are computer-implemented. Each step of the disclosed methods may therefore be performed by a computer. The computer may comprise various combinations of computer hardware, including for example CPUs, RAM, SSDs, motherboards, network connections, firmware, software, and/or other elements known in the art that allow the computer hardware to perform the required computing operations. The required computing operations may be defined by one or more computer programs. The one or more computer programs may be provided in the form of media or data carriers, optionally non-transitory media, storing computer readable instructions. When the computer readable instructions are read by the computer, the computer performs the required method steps. The computer may consist of a self-contained unit, such as a general-purpose desktop computer, laptop, tablet, mobile telephone, smart device (e.g. smart TV), etc. Alternatively, the computer may consist of a distributed computing system having plural different computers connected to each other via a network such as the internet or an intranet.

FIG. 1 depicts a framework for a method of generating EWSs for generating real-time alerts about a patient (e.g. a human or animal subject). Each EWS may, for example, comprise a binary output indicating whether an observation set of a patient is within 24 hours of a composite outcome (unplanned ICU admission, cardiac arrest or mortality). EWSs may be generated at regular intervals based on vital sign information obtained during an assessment time window. The intervals between generation of different EWSs will typically be substantially shorter than the duration of the assessment time window, such that assessment time windows used to generate different EWSs may overlap in time with each other. Alerts are generated in real-time in the sense that they are generated soon after a final input of vital sign information has been obtained that is used to generate the EWS that is used to generate the alert. Each alert may be output before a next EWS is generated. Each alert may be generated dependent on an alerting threshold. For example, when the EWS is higher than an alerting threshold (indicating a higher than normal probability of an imminent adverse event), an alert may be triggered, whereas an alert is not triggered if the EWS is lower than the alerting threshold. The nature of the alert is not particularly limited. The alert could be a visual alert (e.g. a flashing or bold image or text on a display or mobile device) and/or an audio alert (e.g. a ringing alarm).

In an embodiment, the method comprises a step S1 of providing vital sign information. This step may be performed on an ongoing basis during a patient's stay in a medical facility, such as an intensive care unit (ICU). The vital sign information may be input manually by a medical worker via a data entry system (e.g. a computer keyboard or touch screen) or the vital sign information may be provided on an automatic basis by a sensor system 12, as depicted schematically in FIG. 2. The sensor system 12 may comprise a local electronic unit 13 (e.g. a tablet computer, smart phone, smart watch, etc.) and a sensor unit 14 (e.g. a blood pressure monitor, heart rate monitor, etc.). In an embodiment, the vital sign information comprises any one or more of the following components: heart rate (HR); respiratory rate (RR); systolic blood pressure (SBP); diastolic blood pressure (DBP); temperature (TEMP); peripheral capillary oxygen saturation (SPO₂); consciousness level (Alert, Voice, Pain & Unresponsive—AVPU score); and a variable indicating whether supplemental oxygen was provided to the patient at the time of observation.

In step S2, vital sign data is received at a data processing apparatus 5. The vital sign data represents vital sign information obtained in an assessment time window. The assessment time window is typically a period of time ending immediately prior to when the EWS is to be generated. In some embodiments, the assessment time window is a 24 hour period. The vital sign data represents vital sign information obtained at one or more input times within the assessment time window. The vital sign information obtained at each input time may consist of a single component (e.g. a single one of the example components of vital sign information mentioned above, such as a single value representing a measured FIR) or multiple different components (e.g. two or more of the example components of vital sign information mentioned above). In the schematic configuration of FIG. 2, the vital sign data is received by a data receiving unit 8 of the data processing apparatus 5. The data processing apparatus 5 may further comprise a processor 10 configured to carry out steps of the method. The vital sign information may be obtained in a regular or irregular manner during the assessment time window. The vital sign data may thus comprise a time series of data with regular or irregular time intervals between data points and with one or more than one component of vital sign information being provided at each data point.

In step S3, the vital sign data received in step S2 is pre-processed prior to being used as input to a trained recurrent neural network (RNN) in step S4.

An example architecture for the pre-processing is depicted in FIG. 3. In this example, received vital sign data comprises multiple components at each of a plurality of input times. A first subset 301 of the components are sparse continuous variables (e.g. HR, RR, SBP, TEMP and SPO₂) and a second subset 302 of the components are sparse discrete variables (e.g. AVPU and provision of supplemental oxygen).

In some embodiments, Gaussian process regression 303 is applied to continuous variables of the vital sign information (which will typically make up at least a portion of the vital sign information, such as the subset 301 of components in the example of FIG. 3). A Gaussian process model is applied to the continuous variables and used to generate a time series of synthetic vital sign data.

In some embodiments, step function modelling 304 is applied to discrete variables of the vital sign information (e.g. the subset 302 of components in the example of FIG. 3).

The output from the Gaussian process regression 303 and the step function modelling 304 is a posterior mean and a posterior variance for each of the components of the vital sign information processed. As described in further detail below, the posterior mean may be scaled, for example so as to be in the range [−1,1], and the posterior variances may be scaled, for example so as to be in the range [0,1]. Synthetic vital sign data may then be generated at a plurality t of regularly spaced time points (e.g. t=12) to define a feature space 305 to be used as input to step S4 of FIG. 1. Background and example implementation details of the Gaussian process regression 303 and step function modelling 304 are now described in more detail.

### Gaussian Process Regression (GPR)

GPR generalizes multivariate Gaussian distributions to infinite dimensionality and offers a probabilistic and nonparametric approach to model a sparse vital sign time series y as a function of time from admission of a patient to a medical facility (e.g. ICU). In embodiments of the present disclosure, GPR is used to estimate missing observations y*={yᵢ₌₁, . . . , yᵢ₌ₜ} at regularly sampled time steps x*={xᵢ₌₁, . . . , xᵢ₌ₜ}, where t is the number of sampled observations (e.g. the number of time points for the synthetic vital sign data in the assessment window) and the final step xᵢ₌ₜ is the time of observation measured in hours from admission time. In the examples discussed below, t=12 since bi-hourly sampling was performed in a 24 hour assessment window prior to xᵢ₌ₜ.

The smoothness of the model depends on the choice of the covariance function denoted as K. The expected value of the model is determined by the mean function m(x), which in an example implementation is defined as a constant value equal to the vital sign component's mean of the patient population of the same age and sex. Thus,

ƒ(x)·GP(m(x),K(x,x*))

The key assumption of GPR is that y and y* are sampled from the same joint Gaussian distribution, such that

$$[ \begin{matrix}y \\ {y}^{*}\end{matrix} ] \sim N ( y , [ \begin{matrix}K & {K}_{*} \\ {K}_{*}^{T} & {K}_{**}\end{matrix} ] )$$

The covariance matrix in the above equation includes the covariance functions by applying the kernel to our observed and test data,

K representing the similarity measure between all observed values,

K_{*} representing the similarity measure between all observed and test values, and

K_{**} representing the similarity measure between all test values.

Finally, the best estimates for y* and its variance are the mean and variance of the conditional probability p(y*|y), where

p(y*|y)˜N(K_{*}K⁻¹y,K_{**}−K_{*}K⁻¹K_{*}^{T})

In an embodiment, a radial basis function (RBF) with added white noise is adopted as covariance function, such that

$$k ( x , {x}^{′} ) = {σ}_{f}^{2} exp ( - \frac{{( x - {x}^{′} )}^{2}}{2 {l}^{2}} ) + {σ}_{n}^{2} δ ( x , {x}^{′} )$$

where δ(x, x′) is the Kronecker delta function and Θ={l, σ_{f}, σₙ} is the set of hyperparameters. Since it is desired to model vital sign data of the entire patient population, log-normal distributions are applied as priors for the three hyperparameters based on clinical judgment. The model is optimized by minimizing the negative log likelihood with respect to the hyperparameters. The GPR models may be built for example using GPy, which is a GP framework written in python.

### Step Function Modelling

In some embodiments, components of vital sign information that are discrete variables, such as AVPU and provision of supplemental oxygen, are modelled using a piecewise step function ƒ(x)=x where x is the most recent recorded value carried forward. In the detailed examples herein, if the most recent value was unavailable, then a score of 1 (Alert) was assumed for the AVPU score and that supplemental oxygen was not provided so as not to affect the final score.

### Recurrent Neural Network

In some embodiments, step S4 of FIG. 1 is implemented by using the synthetic vital sign data generated in step S3 as input to a trained recurrent neural network (RNN). The trained RNN generates an EWS in step S5. The EWS represents a probability of an adverse event occurring during a prediction time window of predetermined length after the assessment time window. The predetermined length may typically be 24 hours but other predetermined lengths may be used. As explained above, the generated EWS may be used to generate a real-time alert about the patient (e.g. by comparing the EWS to a threshold and initiating an alert, for example a visual or audible alarm, when the threshold is passed).

Due to the assumption of independence and requirement of fixed length inputs in standard feed forward neural networks (FFN), recurrent neural networks (RNNs) have been used for various temporal-based prediction tasks in different levels of health care settings. Given a sequential input, an RNN produces a sequential output at each time step using the current input and the network's previous state.

In some embodiments, the trained RNN particularly comprises a Long Short Term Memory (LSTM) network. LSTM networks develop the concept of the RNN by introducing the concept of the memory cell as the hidden state, as described in general terms in, for example, Hochreiter, S., and Urgen Schmidhuber, J. 1997. Long Short-Term Memory. Neural Computation 9(8):1735-1780.

The inventors have found that a Bidirectional Recurrent Neural Network provides particular improvements. These are described in general terms in, for example, Schuster, M., and Paliwal, K. K. 1997. Bidirectional recurrent neural networks. IEEE Transactions on Signal Processing 45(11):2673-2681.

As depicted schematically in FIG. 4, LSTMs typically contain an input layer 311, a hidden layer 312 and an output layer 313. Given an input of regularly sampled data y*={yᵢ₌₁, . . . , yᵢ₌ₜ}, the hidden layer 312 in an LSTM computes state hₜ at each time point t using the following steps:
A forget gate decides which information is thrown away from the previous cell state:

ƒₜ=σ(W_{ƒ}hₜ₋₁+W_{ƒ}yₜ+b_{ƒ})
An input gate decides which information is stored in the current cell state based on the current input:

iₜ=σ(Wᵢhₜ₋₁+Wᵢyₜ+bᵢ)
The cell state stores which information to forget and store based on the previous two steps:

Cₜ=ƒₜ* Cₜ₋₁+iₜ*tan h(W_{c}hₜ₋₁+W_{c}yₜ+b_{c})
Finally, an output gate modulated by the cell state computes the hidden layer state:

hₜ=σ(Wₕhₜ₋₁+Wₕyₜ+bₕ)* tan h(Cₜ)

where σ is the sigmoid function, W indicates the weights of the respective feed forward neural network, and b is the bias.

As depicted schematically in FIG. 5, a bidirectional LSTM comprises two layers making up the hidden layer 312. The two layers process input from the input layer 311 in forward and reverse directions and yield two hidden layer states h_{t,f} and h_{t,r}.

In some embodiments, the RNN comprises an attention mechanism. An example configuration of an attention mechanism is depicted in FIG. 5, where the average of the two hidden layer states, h_{t,f}, serves as the input to the attention mechanism.

Due to benefits of greater interpretability and extended long-term-dependencies, attention mechanisms (which may also be referred to as attention based models) have been used in various computer vision and natural language processing applications. See, for example, Vaswani, A.; Shazeer, N.; Parmar, N.; Uszkoreit, J.; Jones, L.; Gomez, A. N.; Kaiser, L.; and Polosukhin, I. 2017. Attention Is All You Need. (Nips) and (Vaswani et al. 2017; Xu et al. 2015) or Xu, K.; Ba, J.; Kiros, R.; Cho, K.; Courville, A.; Salakhutdinov, R.; Zemel, R.; and Bengio, Y. 2015. Show, Attend and Tell: Neural Image Caption Generation with Visual Attention.

Attention based models have not previously been used to operate on vital sign information or to provide EWSs.

As shown schematically in FIG. 5, instead of compressing all of the hidden states to compute the final output as in the arrangement of FIG. 4, attention mechanisms allow the model to search the source input and attend to where the most relevant information is available by computing an attention value (which may also be referred to as an attention weight) for every combination of input and output. Further details about attention mechanisms generally may be found in Bandanau, D.; Cho, K.; and Bengio, Y. 2014. Neural Machine Translation by Jointly Learning to Align and Translate. 1-15. Given a regularly sampled input sequence y={yᵢ₌₁, . . . , yᵢ₌ₜ} and its corresponding hidden states h={hᵢ₌₁, . . . , hᵢ₌ₜ} computed by the bidirectional LSTM, the context vector cₜ, output from summing node 312 in FIG. 5, is the weighted combination of the hidden states:

$${c}_{t} = {\sum }_{i = 1}^{t} {α}_{i} {h}_{i}$$

where αᵢ are the weights assigned to the hidden states, such that:

$${α}_{i} = \frac{exp ( {e}_{ij} )}{\sum exp ( {e}_{ij} )}$$

and eₜ is the similarity function

eₜ=a(sᵢ₋₁,hₜ)

where a is considered a feed forward network. The context vector cₜ, output from summing node 312 is provided as input to a dense layer 314 (e.g. a fully connected neural network) which provides a mapping between the context vector cₜ and the output oₜ (e.g. an EWS at a particular time point t). Thus, in embodiments of this type an attention mechanism computes a respective attention weight to apply to a hidden state corresponding to each time point in the assessment time window, and the early warning score is generated via processing of (e.g. via a dense layer 314) a weighted sum of the hidden states weighted by the calculated attention weights (e.g. a context vector).

The generation of the attention weights provides an indication of how the relevance of the input data varies as a function of time. For example, time points in the assessment window having relatively high attention weights indicate a relatively high relevance of those time points to the EWS generated by the RNN. This is demonstrated in the discussion below referring to FIGS. 8-11. The attention weights may be generated independently for different components of the vital sign information and so can provide information of the variation with time of relevance to the generated EWS of each of one or more components of the vital sign information based on the respective computed attention weights.

In some embodiments, the attention weights are learned, for each component of the vital sign information, based on the posterior mean of the component, at each of the time points in the assessment time window. This is the case, for example, in the configuration of FIG. 5.

Configurations of the type depicted in FIG. 5, which uses a combination of an LSTM and an attention mechanism, but without any use of synthetic variances generated by the pre-processing, may be referred to herein as LSTM-ATT systems (where “ATT” stands for attention mechanism).

In some embodiments, the generation of the EWS in step S4 uses the posterior variances generated by the pre-processing of step S3 in addition to the posterior means generated by the pre-processing of step S3. Thus, the mean and variance of each component of the vital sign information generated by the Gaussian process model at each time point tin the assessment window may be used as input to step S4.

Example architectures are depicted in FIGS. 6 and 7. In these embodiments, each posterior mean corresponding to each time point t is used to form an input 321 to a first RNN 331 (e.g. a bidirectional LSTM) and each posterior variance corresponding to each time point t is used for an input 322 to a second RNN 332 (e.g. a bidirectional LSTM). The EWS is generated via processing of outputs from both the first RNN 331 and the second RNN 332 (e.g. by passing the outputs through a dense layer 314 that provides a mapping between those outputs and the EWS). The attention mechanism can be implemented in this context in several ways.

In the example of FIG. 6, the first RNN 331 interacts with an attention mechanism 334. The attention mechanism 334 computes a respective attention weight to apply to a hidden state of the first RNN 331 corresponding to each time point tin the assessment time window. The EWS is then generated using a combination of a weighted sum of the weighted hidden states (weighted by the computed attention weights) of the first RNN 331 and an output from the second RNN 332.

Configurations of the type depicted in FIG. 6, which use a combination of an LSTM and an attention mechanism that learns the attention weights from mean inputs and applies it to the hidden states of the mean and variance inputs, may be referred to herein as UA-LSTM-ATT-1 systems (where “UA” stands for uncertainty aware).

In the example of FIG. 7, the first RNN 331 and the second RNN 332 interact with separate attention mechanisms. Thus, the first RNN 331 interacts with a first attention mechanism 341 and the second RNN 332 interacts with a second attention mechanism 342. The first attention mechanism 341 computes a respective attention weight to apply to a hidden state of the first RNN 331 corresponding to each time point in the assessment time window. The second attention mechanism 342 computes a respective attention weight to apply to a hidden state of the second RNN 332 corresponding to each time point in the assessment time window. Context vectors from each of the first attention mechanism 341 and the second attention mechanism 342 are summed at block 350. The output from block 350 is provided as input to dense layer 314. The dense layer 314 provides a mapping between the summed context vectors and the output of (e.g. an EWS at a particular time point t). Thus, an EWS may be generated using a combination of a weighted sum of the weighted hidden states of the first RNN 331 and a weighted sum of the weighted hidden states of the second RNN 332.

Configurations of the type depicted in FIG. 7, which use a combination of an LSTM and an attention mechanism that learns the attention weights and context vectors from the mean and variance inputs independently, may be referred to herein as UA-LSTM-ATT-2 systems.

### Further Details & Validation

### Dataset

Experiments to valid embodiments were conducted on an anonymized dataset of vital sign observations recorded from adult patients. We included in our model continuous vital signs, such as heart rate (HR), respiratory rate (RR), systolic blood pressure (SBP), diastolic blood pressure (DBP), temperature (TEMP), and peripheral capillary oxygen saturation (SPO2), consciousness level (Alert, Voice, Pain & Unresponsive—AVPU score), and a variable indicating whether supplemental oxygen was provided to the patient at the time of observation. The age and sex of the patient and the timings of unplanned ICU admission, mortality, and cardiac arrest occurrences were also available.

Considering problem as a binary classification task, an event was defined as the composite outcome of the first occurrence of unplanned ICU admission, cardiac arrest or mortality. In the case of multiple occurrences of adverse events, account was taken only of the timing of the first event and observations recorded after an event were removed. Patient episodes were split into a labeled set of event and non-event windows. An event window was defined as an observation measurement of the deterioration and its preceding 24 hours of observations that is within N hours of a composite outcome. A non-event window was defined as an observation measurement and its preceding 24 hours that is not within N hours of a composite outcome. N was set to 24 hours in our study, which is a common evaluation window in the development of EWS systems. We split our dataset to 70% for a training set, 15% validation set and 15% test set. We tested our method on approximately 4,000 observation windows.

### Classification Baselines

The following different classification approaches were compared, where Simple LSTM, LSTM-ATT, UA-LSTM-ATT-1, and UA-LSTM-ATT-2 correspond to the configurations introduced above.
1. NEWS: the clinical benchmark computes a score at each observation step to indicate whether the patient is within 24 hours of an adverse event. We apply NEWS to the raw vital sign data and simply remove observation times with missing data.
2. Simple LSTM: Simple network that produces the probability of an adverse event (e.g. as described above with reference to FIG. 4).
3. LSTM-ATT: Bidirectional LSTM with attention learned from and applied to mean input only (e.g. as described above with reference to FIG. 5).
4. UA-LSTM-ATT-1: the network learns the attention weights from the mean input and applies it to the hidden states of the mean and variance inputs, then sums up the results to compute the final context vector (e.g. as described above with reference to FIG. 6).
5. UA-LSTM-ATT-2: the network learns the attention weights and context vectors from the mean and variance inputs independently and then sums up their two context vectors (e.g. as described above with reference to FIG. 7).

### Problem Setting

Each patient admission has a set of vital sign time series data of 5 continuous variables: HR, SBP, RR, TEMP, and SPO2, and 2 discrete variables: AVPU and the provision of supplemental oxygen, recorded manually at observation times x.
1. We model the 24 hour window preceding each observation time step for continuous vital sign using univariate Bayesian Gaussian Process Regression, whereas each discrete vital sign window is modelled by a piecewise step function (as described above with reference to FIG. 3). We then obtain regularly sampled posterior mean and variance of each vital sign at every two hours up to xᵢ₌ₜ.
2. We scale mean features into the range [−1,1] and variance features into the range [0,1] (as described above with reference to FIG. 3). The scaling and shifting operations are obtained through the training set and then applied to the validation and test sets.
3. For windows shorter than 24 hours, we pre-pad mean values with 0 for both continuous and discrete variables, and variance values with 1 (i.e. maximum uncertainty) for continuous variables only. We do not include variance values for supplemental oxygen and AVPU.
4. We then obtain the final t×m×2 input space 305 (see FIG. 3), where t is the number of time steps, m is the number of vital sign variables per each time step, and 2 corresponds to the mean and variance features for each vital sign. In our study t=12 since we are sampling observations every two hours within a 24 hours window and m=7 corresponding to the number of features considered.
5. Each of the models (Simple LSTM, LSTM-ATT, UA-LSTM-ATT-1, and UA-LSTM-ATT-2) performs binary classification of an event occurring within 24 hours of an observation set at each time step xᵢ₌ₜ.

### Experimental Setup for Validation

GPR Modelling Lognormal priors over the hyperparameters for the vital signs were selected using a combination of a grid-based search and clinical expertise. The lognormal distributions chosen as priors for the radial basis function length scales were (μ=1.0, σ=0.1) for HR, RR, TEMP, and SPO₂ and (μ=1.5, σ=0.1) for SBP and DBP. The lognormal distributions chosen as priors for the radial basis function variance were (μ=0.0, σ=0.1) for HR, SBP, DBP, and SPO₂, (μ=1.5, σ=0.1) for RR, and (μ=3.5, σ=0.1) for TEMP. The lognormal distributions chosen as priors for the Gaussian noise were (μ=0.0, σ=4.0) for HR, SBP, DBP, and SPO₂, (μ=0.0, σ=0.1) for RR, and (μ=1.5, σ=0.1) for TEMP. All GPR models were re-optimized for each of the first five observations, and then once every six new observations, if applicable. Applying lognormal distributions to the three hyperparameters of the GPR enabled us to efficiently model the vital signs of a heterogeneous population.

RNNs All of the RNNs used in step S4 of FIG. 1 were trained for 200 epochs with early stopping using the validation set to avoid overfitting, 50 steps per epoch and a batch size of 50 sequences of the same length. The models were optimized using stochastic gradient descent and Adam optimizer, at a learning rate of 0.01. Each LSTM layer consisted of 12 hidden nodes with L2 regularization. We also used the hyperbolic tangent function as the attention alignment unit.

Performance Evaluation We evaluated the performance using the area under receiver operating characteristics (AUROC) curve, area under precision-recall curve (AU-PR), F1 score, and sensitivity at a generic threshold of 50%, to predict the binary output of a composite outcome. All metrics were evaluated using a bootstrapping technique (number of bootstraps=100). All methods were implemented in Python and Keras.

Table 1 shows the performance results of all models on the testing set. The simple LSTM achieves a lower AUROC of 0.883 [95% CI 0.881-0.885] than the clinical benchmark NEWS, AUROC 0.888 [95% CI 0.886-0.890]. Incorporating the attention mechanism on top of a bidirectional LSTM network improves the mean AUROC from 0.883 to 0.895, and the AU-PR from 0.895 to 0.907. With regards to incorporating uncertainty, the first version of our proposed model UA-LSTM-ATT-1 achieves a comparable performance to LSTM-ATT (AUROC 0.896 [95% CI 0.894-0.898]. However, applying an attention mechanism to the variance input separately achieves the highest mean AUROC of 0.902 [95% CI 0.900-0.903] and the highest mean sensitivity of 0.795 [95% CI 0.792-0.799]. Our model also outperforms NEWS in terms of AU-PR (0.905 vs 0.890) and F1-score (0.814 vs 0.510).

TABLE 1
Models: 1 = NEWS, 2 = LSTM, 3 = LSTM-ATT, 4 = UA-LSTM-ATT-1, 5 = UA-LSTM-
ATT-2; The mean values and confidence intervals were all evaluated using a bootstrapping
technique (nb = 1000) on the test set.

| Model | AU-ROC | AU-ROC CI | AU-PR | AU-PR Cl | FI-Score | Fl-Score CI | Sensitivity | Sensitivity CI |
|---|---|---|---|---|---|---|---|---|
| 1 | 0.888 | 0.886-0.890 | 0.890 | 0.888-0.893 | 0.510 | 0.505-0.514 | 0.348 | 0.344-0.352 |
| 2 | 0.883 | 0.881-0.885 | 0.895 | 0.893-0.897 | 0.800 | 0.797-0.802 | 0.772 | 0.769-0.776 |
| 3 | 0.895 | 0.893-0.897 | 0.907 | 0.905-0.909 | 0.815 | 0.812-0.817 | 0.774 | 0.771-0.778 |
| 4 | 0.896 | 0.894-0.898 | 0.902 | 0.900-0.094 | 0.809 | 0.807-0.812 | 0.769 | 0.765-0.772 |
| 5 | 0.902 | 0.900-0.903 | 0.905 | 0.903-0.908 | 0.814 | 0.812-0.817 | 0.795 | 0.792-0.799 |

To further investigate the effect of incorporating the uncertainty of the data, we visualize the attention weights learned from and applied to the mean function in the UA-LSTM-ATT-2, which achieved the highest AUROC, and the LSTM-ATT model in FIGS. 8-11. The curves 201-207 correspond to the variation of relevance with time for different components of the vital sign information as follows: AVPU (201), Supplemental oxygen (202), HR mean (203), SBP mean (204), TEMP mean (205), SPO₂ mean (206) and RR mean (207). The LSTM-ATT distributes the attention weights more uniformly across the window in comparison to UA-LSTM-ATT-2, which exerts higher attention more distinctly on a selected subset of time period (indicated by darker shading and labelled 210). These time periods of higher attention indicate greater relevance to the generated EWS and may provide useful information to a medical worker interpreting the generated EWS.

We also compare the performance of LSTM (dot chain line), LSTM-ATT (broken line), and UA-LSTM-ATT-2 (solid line) for sequences of different lengths in FIG. 12. The figure suggests that UA-LSTM-ATT-2 outperforms LSTM-ATT for shorter sequences, and implies that LSTM-ATT performs well with longer sequences. Performance of all models improved as the sequence length increased.

Based on an alerting threshold of 0.5, we applied a multinomial logistic regression to classify four classes where windows were (1) True Positive (TP) in UA-LSTM-ATT-2 and False Negative (FN) in NEWS (22.6%), (2) TP in NEWS and FN in UA-LSTM-ATT-2 (0.048%), (3) True Negative (TN) in UA-LSTM-ATT-2 and False Positive (FP) in NEWS (0.048%), and (4) TN in NEWS and FP in UA-LSTM-ATT-2 (7.5%). Diagnosis codes, grouped by official ICD-10 guidelines (ICD), was considered a significant predictor variable (p<0.05) in distinguishing Class 1 and 4 only. With the primary objective of alerting for deteriorating patients, UALSTM-ATT-2 improved the alerting performance, defined as the ratio of class 1 windows to FN in NEWS, for several diagnosis groups as shown in Table 2, reaching up to 84.3% improvement for patients with diseases of the respiratory system.

TABLE 2
Alerting improvement of UA-LSTM-ATT-2 over
NEWS in identifying event windows
of patients with specific diseases at an alerting
thresholds of 0.5. Results are shown for diagnosis
groups with at least 250 event windows.

| | No. of | No. of | Alerting |
|---|---|---|---|
| Diagnosis | windows | admissions | Improvement |
| Group | (%) | (%) | % |
| Certain Infectious and | 250 (6.0) | 25 (5.1) | 66.0 |
| Parasitic Diseases | | | |
| Neoplasms | 443 (10.5) | 53 (10.8) | 80.4 |
| Diseases of the circulatory | 565 (13.5) | 76 (15.4) | 63.5 |
| system | | | |
| Diseases of the respiratory | 784 (18.7) | 122 (24.7) | 84.3 |
| system | | | |
| Diseases of the digestive | 502 (12.0) | 72 (14.6) | 68.6 |
| system | | | |
| Diseases of the | 271 (6.5) | 22 (4.5) | 78.6 |
| musculoskeletal system | | | |
| and connective tissue | | | |
| Diseases of the | 274 (6.5) | 38 (7.7) | 50.6 |
| genitourinary system | | | |
| Injury, poisoning and | 766 (18.2) | 48 (9.7) | 47.9 |
| certain other consequences | | | |
| of external causes | | | |

FIGS. 13 and 14 compare performance of UA-LSTM-ATT-2 (solid line) with NEWS (broken line). FIG. 13 shows variation of a mean probability of an event (averaged over calculations of EWS taken at multiple times) determined using the respective models for non-deteriorating patients in a sample hospitalization window. Both models consistently output low probabilities, as expected for non-deteriorating patients. FIG. 14 shows variation of a means probability of an event (averaged over calculations of EWS taken at multiple times) determined using the respective models for deteriorating patients in the 24 hours leading up to an event, with the event occurring at time=0 hours on the horizontal axis. Both models consistently output relatively high probabilities but the probability are consistently higher for UA-LSTM-ATT-2 and show a more marked rise towards the event, suggesting that the UA-LSTM-ATT-2 performs better than NEWS.

### Further Embodiments

Methodology of the type described above can be adapted to take account of supplementary information in addition to the vital sign information. The supplementary information may comprise a diagnosis code (e.g. an ICD-10 diagnosis code—the 10^{th} revision of the International Statistical Classification of Diseases and Related Health Problems, ICD, a medical classification list by the World Health Organisation see below) representing a diagnosis of the patient at a time of admission of the patient to a medical facility. Alternatively or additionally, the supplementary information may comprise laboratory test data. Embodiments described below explain how such information can be fused with information obtained from vital sign data in order to provide an improved alert. Embodiments described below also include a variation on how the recurrent neural network can be configured to provide an early warning score. The overall model described below is referred to as iFEWS in the present disclosure.

The problem of detecting clinical deterioration may be considered as a binary classification task. For each component of vital sign information recorded for a patient, a model (e.g. iFEWS) may be provided that predicts the probability of a composite outcome (e.g. represented as an early warning score) within the next N hours. Each component of vital sign information may be considered as an event or non-event window D_{W}=[xᵢ,yᵢ]ᵢ₌₁ⁿ, with N=24 hours for example. As will be described in further detail below, laboratory test data may also be taken into account. Laboratory test data may be represented as a vector of the most recently-measured laboratory tests D_{L}=[xₗ,z] in the last k days for example. As will be described in further detail below, diagnosis codes may also be taken into account. The diagnosis codes may include a first ICD-10 diagnosis code d assigned to the patient at admission for example. In this case, d is a categorical variable. The model may then estimate the posterior probability l of being within N hours of an adverse outcome, such that l∈[0,1].

The performance of deep learning models depends on the representation of the input data. It is therefore desirable to learn an efficient representation of the explanatory features of the data, which can then be used for subsequent predictive tasks. The data available for calculating early warning scores considered in the present disclosure can be heterogeneous in nature, ranging from both dense and sparse time-series variables, such as vital signs and laboratory tests, respectively, to discrete categorical variables such as diagnosis codes. The different variables may be treated based on how and when they were collected relative to the point of prediction as will be described below. A model may then be trained by learning an efficient representation of each variable type (e.g. using an autoencoder for the vital sign information) before combining those representations for our classification task. We now describe example data pre-processing and learning techniques for each variable type (i.e. vital sign data, laboratory test data and diagnosis codes).

### Vital Sign Data Pre-Processing

As described earlier, since the vital signs are irregularly sampled, a Gaussian process model may be used to generate a time series of synthetic vital sign data at each of a plurality of regularly spaced time points in an assessment time window. This may be done by first applying a patient-specific feature transformation for each window using Gaussian process regression (GPR) with a squared-exponential kernel to obtain equally sampled posterior mean and variance estimates. The squared-exponential kernel has been shown to be suitable for modelling physiological data. These posterior mean and variance estimates are concatenated for all the vital signs to obtain: Y_{μ}=[y_{μ,j}]ⱼ₌₁^{m} and Y_{σ}=[y_{σ,j}]ⱼ₌₀^{m}, where Y_{μ}, Y_{σ}∈ [symbol] ^{m×T} and y_{μ,j} and y_{σ,j} are the GPR mean and variance for the jth vital sign, such that j=1, . . . , m.

### Multi-Channel Autoencoder

As described earlier, a recurrent neural network may be used to generate an early warning score using the generated synthetic vital sign data. In the present embodiment, the recurrent neural network forms part of an autoencoder 400. An example of such a configuration is depicted schematically in FIG. 15. Use of the configuration to generate a composite early warning score using additional early warning scores based on a diagnosis code and based on laboratory test data in the overall iFEWS model is depicted in FIG. 16.

An autoencoder learns an efficient lower-dimensional representation of the (higher-dimensional) data through unsupervised learning. The basic architecture consists of an encoder 406 that learns a compact latent representation Lᵥ from the input data 404, and a decoder 410 that reconstructs the input data 404 using the latent representation Lᵥ (to provide reconstructed input 412). In embodiments of this type, the early warning score is generated using the latent representation from the autoencoder 400.

In an embodiment, as exemplified by FIG. 15, the autoencoder 400 comprises multiple encoder channels 406. Each encoder channel 406 receives vital sign data 404 representing a different component of vital sign information. In the example of FIG. 15, three encoder channels 406 are depicted for illustrative purposes but more encoder channels 406 could be provided (one for each different component of vital sign information available in the input data).

In an embodiment, each encoder channel 406 comprises an attention mechanism 408. Each attention mechanism is configured to compute a context vector. The latent representation Lᵥ is obtained by combining the context vectors from the multiple encoder channels 406 and associated attention mechanisms 408.

As a specific example, a joint latent representation Lᵥ of m components of vital sign information may be jointly reconstructed using a multi-channel attention-based autoencoder 400 that consists of m attention-based encoders 406 and a single decoder 410, in accordance with the architecture shown in FIG. 15. A single-channel encoder E_{μ,j} first processes a vital-sign sequence j independently using a recurrent neural network (e.g. a bidirectional Long Short Term Memory network, as described earlier) in order to maximise information retrieval in the forward and backward directions. The average of the forward and backward hidden-state outputs for vital sign component j,h̅_̅{̅t̅,̅j̅}̅, is then processed using an attention-based block A_{μ,j} to encode interpretability and compute the context vector:

$${c}_{j} = {\sum }_{t = 1}^{T} {α}_{t , j} \overline{{h}_{t , J}}$$

The context vectors of the m vital signs are concatenated to obtain the latent representation Lᵥ:

Lᵥ=[c₁^{T}, . . . ,cₘ^{T}]

In an embodiment, the autoencoder 400 comprises a single decoder channel 410. The single decoder channel 410 may comprise plural layers. In the example shown the decoder channel 410 comprises three dense layers. The decoder channel 410 outputs a reconstructed input 412 corresponding to each of the encoder channels 406.

In an embodiment, the latent representation Lᵥ is mapped by applying a sigmoid function to obtain the reconstructed input 412 of all vital signs ŷ:

ŷ=σ(W₄g₃(W₃g₂(W₂g₁(W₁Lᵥ+b₁)+b₂)+b₃)+b₄)

where W₁, W₂, and W₃ are the weight matrices and b₁, b₂, and b₃ are the bias vectors of the dense layers of the decoder channel 410. W₄ is the weight matrix and b₄ is the bias vector of the final sigmoid layer. The activation functions of the dense layers are g₁, g₂, and g₃.

In an embodiment, the parameters of the autoencoder 400 are optimised by minimising a binary cross-entropy loss for all of the encoder channels 406 (i.e. for each of the components of vital sign information):

$${ℒ}_{RL} ( y , \hat{y} ) = - \frac{1}{m \times T} {\sum }_{i = 1}^{m \times T} {y}_{i} \cdot log ( {\hat{y}}_{i} ) + ( 1 - {y}_{i} ) \cdot log ( 1 - {\hat{y}}_{i} )$$

where m×T is the total number of input features from all of the vital-sign components.

In an embodiment, the latent representation Lᵥ is further processed (in the block labelled σᵥ in FIG. 16) using a multi-layer perceptron with a final sigmoid layer to provide an early warning score based on the vital sign data lᵥ (a probability of deterioration):

lᵥ=σ(WᵥLᵥ+bᵥ)

where Wᵥ is the weights matrix and bᵥ is the bias vector. This component of the iFEWS model may be denoted as MC-AE-ATT-CLᵥ, corresponding to the multichannel autoencoder with attention (MC-AE-ATT) with subsequent (-CLᵥ) classification of the latent representation.
Learning from Laboratory Test Data

As mentioned above, laboratory test data may be used to improve a generated early warning score. Thus, the methods described above may be adapted to additionally provide the step of receiving laboratory test data. The laboratory test data represents information obtained from one or more laboratory tests performed on the patient. In an embodiment, the laboratory test data comprises measurement results relating to one or more of the following components: Haemoglobin (HGB), which is the number of red blood cells that transport oxygen to the body organs and carry back carbon dioxide to the lungs, measured by a blood test; White Blood Cells (WBC), or leukocytes, which are counted in blood tests to help detect infection that the immune system is trying to fight; Sodium (Na) test, which is a blood test that measures the amount of sodium in the blood, an electrolyte that regulates the amount of water surrounding the cells and maintains blood pressure; Potassium (K), which is also an electrolyte that is vital for regulating fluid volumes in cells and blood pH, Albumin (ALB), which is a protein made by the liver that prevents fluid in the bloodstream from leaking; Urea (UR), measured by urine or blood tests, is the metabolic waste product of protein breakdown; Creatinine (CR), which is a waste product generated by the breakdown of muscle tissue that specifically indicates kidney function; Hematocrit (HCT), which measures the proportion of red blood cells in the total blood count; Bilirubin (BIL), which is a yellow pigment in the blood that is produced by the breakdown of red blood cells—it is used as an indicator of anaemia, jaundice or liver disease; Troponin (TROP), which are proteins in the blood that measure contractions in the heart muscle; C-Reactive Protein (CRP), which is an acute-phase protein released by the liver after tissue injury, such as sepsis or strokes, that indicates degree of infection or inflammation.

In comparison to vital signs, laboratory tests are normally less frequently measured. In embodiments of the present disclosure, the laboratory test data may be pre-processed to yield a real-time alerting score as provided using the vital sign data (as described above). In an exemplary approach, each of one or more of the components of vital sign information is associated with a most recently-collected set of laboratory test data D_{L}=[xₗ,z] during the previous N×k hours, where k is the number of days, xₗ is the time the laboratory tests were measured, and z∈ [symbol] ^{q} is a vector of q (scalar-valued) laboratory-test measurements. The time between a vital-sign measurement and the laboratory test measurements is denoted as tᵥ₋ₗ=xₙ−xₗ, where xₙ is the time of prediction based on the vital-sign measurements. Physiologically implausible and missing values were replaced by the mean of the respective variable in the training set and the features were then scaled to obtain the final feature set \hat{z}.

A trained model of a relationship between laboratory test data and probabilities of an adverse event occurring during the prediction time window is used to generate an early warning score based on the laboratory test data. In an embodiment, the trained model comprises a logistic regression model. The use of a logistic regression model makes it possible to assess the learned coefficients assigned to each component (variable) of the laboratory test data. In the block labelled σₗ in FIG. 16, the model generates an early warning score iₗ based on the laboratory test data (a probability of deterioration) as follows:

lₗ=σ(Wₗ\hat{z}+bₗ)

where Wₗ is the weights matrix, \hat{z} is the vector of processed laboratory tests, and bₗ is the vector of biases. This module may be denoted with the suffice -CLₗ.

A composite early warning score may be obtained using a combination of at least the early warning score lᵥ generated using the trained recurrent neural network (based on the vital sign data) and the early warning score lₗ based on the laboratory test data. An example implementation is described in further detail below with reference to FIG. 16. An alert may be generated using the composite early warning score. As will be demonstrated below, taking account of the laboratory test data improves the generation of the alert (e.g. by reducing false positives without losing sensitivity).

In an embodiment, the model of the relationship between laboratory test data and probabilities of an adverse event includes a decay term to model an effect of delay between obtaining of the laboratory test data and a time at which the composite early warning score is to be obtained. This may be implemented for example by accounting for a time difference between the vital-sign measurements and the laboratory-test measurements tᵥ₋ₗ by further processing lₗ using an exponential decay model (depicted as block 420 in FIG. 16), such that an updated early warning score \hat{l}ₗ (which may also be referred to as an updated label) is obtained as follows:

$${\hat{l}}_{l} = {l}_{l} exp - λ \frac{{t}_{v - l}}{Nk}$$

where λ is learned during training of the model. This equation adjusts the posterior probability of an outcome computed using the laboratory tests using the exponential decay model.

As validation of this approach the inventors considered two sets of laboratory tests as input variables: (1) set S consisting of 8 laboratory tests; and (2) set U consisting of 4 additional laboratory-test variables. (Set S∪U therefore contains 11 variables in total). The results are discussed below.

Learning from Diagnosis Code Data

As mentioned above, diagnosis codes may be used to improve the generated early warning score. Thus, the methods described above may be adapted to additionally provide the step of receiving a diagnosis code (alternatively or additionally to receiving laboratory test data). In an embodiment, the diagnosis code represents information representing a diagnosis of the patient made at a time of admission of the patient to a medical facility.

In some embodiments, the diagnosis code is provided in a standard format, such as the ICD-10 format. Each diagnosis code may consist of several characters that represent a particular disease or illness. In an embodiment, diagnosis codes were grouped into 21 groups based on the high-level grouping of the ICD-10 codes. An additional group was created to represent missing or incorrect diagnosis codes that do not map to the ICD-10 dictionary. Thus, in total there were 22 possible diagnosis categories. To learn a representation of the discrete diagnosis codes, we incorporated an embedding module 422 (depicted in FIG. 16) with a non-negativity constraint. The embedding module 422 thus maps each discrete code d into a latent vector of positive real numbers \hat{d}. In the block labelled σ_{d} in FIG. 16, the latent vector \hat{d} is then used to generate an early warning score l_{d} based on the diagnosis code (a probability of deterioration) as follows:

l_{d}=σ(W_{d}l_{d}+b_{d})

where W_{d} is the weights matrix and b_{d} is the bias vector. Thus, a trained model of a relationship between diagnosis codes and probabilities of an adverse event occurring during the prediction time window is used to generate an early warning score based on the diagnosis code.

A composite early warning score may be obtained using a combination of at least the early warning score lᵥ generated using the trained recurrent neural network (based on the vital sign data) and the early warning score l_{d} based on the diagnosis code. In some embodiments, a composite early warning score is obtained using a combination of the early warning score lᵥ generated using the trained recurrent neural network (based on the vital sign data), the early warning score l_{d} based on the diagnosis code, and the early warning score lₗ based on the laboratory test data (optionally updated as described above to give \hat{l}ₗ). An example implementation is described in further detail below with reference to FIG. 16. An alert may be generated using the composite early warning score. As will be demonstrated below, taking account of the diagnosis code improves the generation of the alert (e.g. by reducing false positives without losing sensitivity).

### Generation of Composite Early Warning Score

FIG. 16 depicts computation of a final output l, which may be referred to as a composite early warning score. The composite early warning score is computed in block σₒ in this example using all three auxiliary outputs from the three separate channels in FIG. 16: the early warning score lᵥ from the vital sign data, the time-adjusted early warning score \hat{l}ₗ from the laboratory test data, and the early warning score l_{d} from the diagnosis code, such that

l=σ(Wₒ[lᵥ;\hat{l}ₗ;l_{d}]+bₒ)

As described above, the three different types of input are first processed with different feature learning techniques to compute the three separate early warning scores (l_{d}, \hat{l}ₗ, and lᵥ). The final output l is then computed to indicate the probability of an occurrence of a composite outcome within the next N hours of a vital-sign measurement.

### Continued Training

In comparison with data encountered in computer vision and natural language processing, clinical datasets tend to be smaller in magnitude. To address this, in some embodiments a performance of the iFEWS model is improved by first pre-training its components independently and then fine-tuning their parameters as part of the larger model. In an embodiment, the model may be trained in a two-fold process. First, the MC-AE-ATT component is pre-trained independently by minimizing the binary cross-entropy loss described above. Secondly, the CLₗ component is pre-trained independently by minimizing the binary cross-entropy loss but with a newly defined output \hat{l}∈(0,1), which indicates the probability of an adverse event at any time in the future during the current admission.

The pre-trained weights of MC-AE-ATT and CLₗ components may then be used to initialise their corresponding weights in the iFEWS model. The classification objective of iFEWS is the binary cross-entropy loss of the true labels (early warning scores) \tilde{l} and the predicted labels (early warning scores):

$${ℒ}_{CL} ( \tilde{l} , l ) = - \frac{1}{N} {\sum }_{i = 0}^{N} {\tilde{l}}_{i} \cdot log ( {l}_{i} ) + ( 1 - {\tilde{l}}_{i} ) \cdot log ( 1 - {l}_{i} )$$

where N is the number of training samples.

The final objective function of iFEWS consisted of the joint loss function:

[symbol]

_{JT}= [symbol] _{RL}(y,ŷ)+ [symbol] _{CL}(\tilde{l},l)

We included the reconstruction loss function of the MC-AE-ATT component, since it contains the majority of parameters that compute the latent representation of the vital-sign measurements. (We note that losses [symbol] _{RL} and [symbol] _{CL} could be combined in the affine β [symbol] ₐ+(1−β) [symbol] _{b}, and β=0.5 performed best empirically for our task.)

### Model Variants as Baselines

To evaluate the effect of the design choices on the overall performance of the model, and to justify model complexity, we assess several simpler variants of iFEWS. For learning the representation of the vital signs, we first developed and evaluated a single-channel autoencoder (SC-AE) that simply concatenated all the vital-sign sequences as one input. The inputs were then processed by three dense layers. In order to encode temporal information, we then designed the multichannel autoencoder (MC-AE) that processed each vital-sign sequence independently using an BiLS™ network. Since the BiLS™ network lacks interpretability, we finally incorporated the attention mechanism in each channel (MC-AE-ATT).

We also compared the iFEWS model to LDTEWS and LDTEWS:NEWS as standard clinical benchmarks. Both LDTEWS and LDTEWS:NEWS only included 8 routinely collected laboratory tests (i.e. Hb, WCC, U, ALB, CR, NA, and K) as included in set S. We further included TROP, HCT, TBIL, and CRP in set U and evaluated our deep learning models using both sets.

### Evaluation Metrics

We evaluated the performance of our models using several metrics based on the respective task. For the autoencoders, we measured the mean squared error (MSE) to assess the reconstruction quality.

During model development and validation, we assessed the model variants and components using AUROC and AUPRC. For our proposed iFEWS model and other classifiers, we used the AUROC, sensitivity, specificity, and PPV evaluated on the testing sets. All metrics were performed using a bootstrapping technique with replacement with a fixed number of bootstraps (nb). We compared the performance of the models across patients aged 16-45 years and >45 years, and across three outcomes (unplanned ICU admission, cardiac arrest, and mortality) independently.

### Deep Learning Experiments

All hyperparameters of the model were optimised empirically using a balanced training and validation set, referred to as D_{O,1B}. The regularly-spaced mean vital-sign measurements (y_{μ}) were transformed with min-max scaling of [0,1]. All of the vital-sign autoencoder models were trained with 20 epochs, with early stopping by monitoring the loss on the validation set. The encoder module of the SC-AE consisted of four dense layers with 64 nodes followed by a latent-space dense layer consisting of 12 nodes. The decoder module of the SC-AE consisted of four dense layers with 64 nodes and a final sigmoid layer with 84 output nodes (corresponding to the 12 equidistant timesteps of the 7 vital signs). The encoder of the MC-AE model consisted of a BiLS™ with 5 output nodes at each timestep and the decoder consisted of four dense layers with 64 nodes each. The classifier consisted of five dense layers and a final sigmoid layer.

To assess the predictive power of vital signs and the continued learning scheme, we trained MC-AE-ATT-CLᵥ independently using three different training schemes. The first training scheme involved pre-training MC-AE-ATT independently, and then fixing its weights during the training of the latent space classifier -CLᵥ. The second scheme involved joint training of MC-AE-ATT and the latent space classifier -CLᵥ with random initialisation of weights. The third scheme, continued learning, involved pre-training the MC-AE-ATT independently followed by joint learning with the latent space classifier -CLᵥ.

The laboratory-test measurements were transformed using standardisation with a zero mean and unit variance. For the models using laboratory tests, we trained and evaluated our models for the original label \tilde{l} (i.e. the vital-sign measurements are within N hours of an outcome). The models were trained with 100 epochs with early stopping by monitoring the classification loss on the validation set in order to avoid overfitting.

The diagnosis codes embedding module performed best when it computed 3-dimensional vector representations. We also compared embeddings to one-hot encoding, and we found that (in experiments not shown here for brevity) that the model using embeddings performed better. We also did not pre-train the embedding in the continued learning training scheme because it did not show any predictive power when learning in isolation of components of the larger models Weights that were not pre-initialised with the continued learning scheme were randomly initialised. All the models were optimised using the Adam optimiser and implemented using Keras (v 2.2.2) (a high-level neural networks API—www.keras.io) with a TensorFlow backend (v 1.5.0—www.tensorflow.org).

### Feature Learning of Vital Signs

The reconstruction errors in terms of the MSE of the vital-sign sequences in the training set D_{O,1B} and testing sets D_{O,2} and Dₚ are shown in Table A.

TABLE A
Mean and standard deviation of the mean squared error on the training
set D_{O,1B} and testing sets D_{O,2} and D_{P} using the different autoencoder
architectures for reconstructing all vital signs.
All values are on a scale of 10⁻³.

| Model | [symbol] _{O,1B} | [symbol] _{O,2} | [symbol] _{P} |
|---|---|---|---|
| SC-AE | 2.00 ± 3.60 | 1.08 ± 2.06 | 2.24 ± 15.66 |
| MC-AE | 2.00 ± 4.69 | 1.39 ± 2.79 | 2.49 ± 15.27 |
| MC-AE-ATT | 4.14 ± 6.32 | 2.72 ± 4.45 | 3.70 ± 16.17 |

The MSE increases as the model complexity increases across all datasets. While MC-AE-ATT is the most interpretable since it incorporates an attention mechanism, it yields the highest reconstruction error in all datasets. Additionally, D_{P} has the highest standard deviation of errors across the three datasets. This may be because the vital-sign sequences in D_{P} were scaled using transformations learned from an independent and foreign dataset D_{O,1B} On the other hand, D_{O,1B} and D_{O,2} belong to the same distributions as they were both obtained from the same hospital source.

Table B presents the performance of the different training schemes on a validation set D_{O,V}.

TABLE B
Performance on the validation set D_{O,V} using the MC-AE-ATT with classification of the
latent space (−CLᵥ) and the respective numbers of trainable and non-trainable parameters. Mean
and confidence intervals were evaluated using a bootstrapping technique (nb = 1,000).

| | Trainable | Non-trainable | | |
|---|---|---|---|---|
| Training Scheme | parameters | parameters | AUROC | AUPRC |
| Pre-initialisation | 12,147 | 33,775 | 85.8 (85.7-85.8) | 86.4 (86.3-86.4) |
| Joint Learning | 45,992 | 0 | 88.0 (87.9-88.0) | 89.2 (89.2-89.2) |
| Continued-Learning | 45,992 | 0 | 89.4 (89.3-89.4) | 87.9 (87.8-87.9) |

Pre-initialisation has the lowest number of trainable parameters, since it only involves training of the latent space classifier. It also achieves the lowest AUROC [95% CI 85.7-85.8] and AUPRC [95% 86.3-86.4] values across all schemes. Continued learning achieves the highest AUROC [95% 89.3-89.4] across all schemes; we choose to adopt it for training our overall model. We note that the AUPRC values are considerably high since the validation set D_{O,V} is balanced as is the training set from which it was derived.

### Predictive Power of Laboratory Tests

Table C summarises the performance of LDTEWS and the LR models on the validation set D_{O,1V} using the two sets of laboratory-test variables, S and U.

TABLE C
Performance evaluation of simple logistic regression using
laboratory tests in comparison to the clinical baseline
(LDTEWS) on the validation set D_{O,1V}. Note that S denotes the
set of variables considered in LDTEWS and U denotes the set including
four additional laboratory tests. Mean and confidence intervals
were evaluated using a bootstrapping technique (nb = 1,000).

| Model | AUROC | AUPRC |
|---|---|---|
| LDTEWS_{S} | 67.2 (67.1-67.2) | 67.4 (67.3-67.4) |
| LR_{S} | 70.3 ( 70.3-70.3) | 71.2 (71.2-71.3) |
| LR_{U} | 72.7 (72.6-72.8) | 73.6 (73.5-73.7) |

LDTEWS achieves the lowest performance for both labels in terms of AUROC [95% CI 67.1-67.2] and AUPRC [95% CI 67.3-67.4]. We also observe that LR achieves the highest AUROC [95% 72.6-72.8] and AUPRC [95% CI 73.5-73.7] when using the laboratory-tests dataset U. This suggests that incorporating the additional variables in set U over set S improves the predictive performance of a laboratory-tests based classifier.
Performance Evaluation of iFEWS

Table D summarises the performance results of the final models on D_{O,2}.

TABLE D
Performance evaluation of the different classifiers on D_{O,2}. The decision threshold of all
classifiers was adjusted to achieve a specificity similar to that of NEWS (≈89.0). The subscripts
indicate (i) what types of features were used in the LR model and (ii) the type of autoencoder in
iFEWS. Mean and confidence intervals were evaluated using a bootstrapping technique (nb = 1,000).

| Model | AUROC | Sensitivity | Specificity | PPV |
|---|---|---|---|---|
| i.i.d | | | | |
| NEWS | 86.6 (86.5-86.6) | 70.2 (70.2-70.3) | 88.8 (88.8-88.8) | 5.0 (5.0-5.0) |
| LDTEWS:NEWS | 88.4 (88.4-88.4) | 73.8 (73.7-73.8) | 88.8 (88.8-88.8) | 5.2 (5.2-5.2) |
| LR_{L,V,D} | 88.8 (88.8-88.8) | 74.7 (74.6-74.8) | 89.0 (88.9-89.0) | 5.3 (5.3-5.4) |
| Model Vartiants | | | | |
| iFEWS_{SC-AE} | 89.0 (89.0-89.7) | 74.8 (74.7-74.9) | 88.9 (88.9-88.9) | 5.3 (5.3-5.4) |
| iFEWS_{MC-AE} | 90.2 (90.2-90.2) | 76.2 (76.2-76.3) | 88.8 (88.8-88.8) | 5.4 (5.4-5.4) |
| iFEWS_{MC-ATT-AE} | 90.0 (90.0-90.0) | 77.0 (77.0-77.1) | 88.7 (88.7-88.7) | 5.4 (5.4-5.4) |

iFEWS and a variant of iFEWS without attention (iFEWS_{MC-AE}) achieved the highest AUROC values, [95% CI 90.0-90.0] and [95% 90.2-90.2] respectively. iFEWS also had the highest sensitivity [95% CI 77.0-77.1]. With respect to the clinical baseline that is adopted in practice, NEWS, our model is approximately 4% higher. iFEWS_{SC-AE} achieved the lowest AUROC [95% CI 89.6-89.7] across the three autoencoder models. Despite MC-AE-ATT having the highest reconstruction error (as shown in Table A), the performance of iFEWS is comparable with that of iFEWS_{MC-AE}. This suggests that incorporating an attention mechanism improves interpretability while maintaining model performance. All models achieved a comparable PPV.
Table E shows the performance of iFEWS on sub-populations in D_{O,2}.

TABLE E
Performance evaluation of iFEWS in comparison to LDTEWS:NEWS across sub-
populations of interest i.e. 16-45 years old, >45 years old, and each of the three events in the
composite outcome, in D_{O,2}. The adjusted decision threshold for iFEWS was 0.63, to achieve a
similar overall specificity of the clinical benchmark NEWS (≈89.0). Mean and confidence
intervals were evaluated using a bootstrapping technique (nb = 1,000) for the respective sub-
population.

| Model | AUROC | Sensitivity | Specificity | PPV |
|---|---|---|---|---|
| 16-45 year: old | | | | |
| iFEWS | 87.2 (87.1-87.4) | 56.5 (56.1-56.9) | 94.0 (94.0-94.0) | 3.1 (3.0-3.1) |
| LDTEWS:NEWS | 81.7 (81.5-81.9) | 50.6 (50.2-51.0) | 94..4 (94.4-94.4) | 2.9 (2.0-3.0) |
| >45 years old | | | | |
| iFEWS | 90.0 (89.9-90,0) | 78.3 (78.2-78.3) | 87.9 (87.9-87.9) | 5.6 (5.6-5.6) |
| LDTEWS:NEWS | 88.6 (88.6-88.6) | 75.2 (75.1-75.3) | 87.9 (87.9-87.9) | 5.4 (5.4-5.4) |
| Unplanned ICU | | | | |
| iFEWS | 84.9 (84.8-85.0) | 63.0 (62.9-63.2) | 90.1 (90.1-90.1) | 1.5 (1.5-1.5) |
| LDTEWS:NEWS | 80.1 (80.0-80.2) | 55.8 (55.6-56.0) | 90.2 (90.2-90.2) | 1.3 (1.3-1.3) |
| Cardiac Arrest: | | | | |
| iFEWS | 80.9 (80.8-81.1) | 53.0 (52.7-53.3) | 90.2 (90.2-90.2) | 0.4 (0.3-0.4) |
| LDTEWS:NEWS | 78.3 (78.2-78.5) | 51.3 (51.0-51.6) | 90.2 (90.2-90.2) | 0.3 (0.3-0.3) |
| Mortality | | | | |
| iFEWS | 93.8 (93.6-93.8) | 85.8 (85.7-85.9) | 88.8 (88.8-88.8) | 4.1 (4.1-4.1) |
| LDTEWS:NEWS | 93.7 (93.6-93.7) | 84.1 (84.0-84.2) | 88.8 (88.8-88.9) | 4.0 (4.0-4.0) |

Across the younger patients, iFEWS achieved a higher AUROC than LDTEWS:NEWS, [95% CI 87.1-87.4] and [95% CI 81.5-81.9] respectively. The performance of iFEWS for 16-45 years old patients is also superior to that of a supervised learning model DEWS (AUROC [95% CI 81.8-82.2]) and NEWS (AUROC [95% CI 75.7-76.2]). This represents more than 10% increase relative to the performance of the current state-of-the-art (i.e. NEWS) for the young patient group. For the group of elder patients, for unplanned ICU admission, and for mortality, iFEWS consistently performed better than LDTEWS:NEWS in terms of the AUROC. For mortality, iFEWS achieved a similar AUROC to LDTEWS:NEWS, [95% CI 93.6-93.7] and [95% CI 93.6-93.7] respectively. However, iFEWS had a higher sensitivity, [95% CI 85.7-85.9] compared to [95% CI 84.0-84.2].
Table F presents the performance of iFEWS across the different patient sub-populations in D.

TABLE F
Performance evaluation of iFEWS in comparison to LDTEWS:NEWS across sub-
populations of interest i.e. 16-45 years old, >45 years old, and each of the three events in the
composite outcome, in D_{P}. The adjusted decision threshold for iFEWS is 0.63 to achieve a similar
overall specificity of the clinical benchmark NEWS (≈89.0). Mean and confidence intervals were
evaluated using a bootstrapping technique (nb = 1,000) for the respective sub-population

| Model | AUROC | Sensitivity | Specificity | PPV |
|---|---|---|---|---|
| Overall | | | | |
| iFEWS | 89.5 (89.5-89.5) | 73.3 (73.3-73.4) | 89.5 (89.5-89.5) | 7.0 (7.0-7.0) |
| LDTEWS:NEWS | 88.6 (88.5-88.6) | 63.1 (63.4-68.5) | 90.9 (90.9-90.9) | 7.5 (7.5-7.5) |
| 16-45 years old | | | | |
| iFEWS | 94.3 (94.2-94.3) | 76.1 (75.8-76.4) | 94.2 (94.2-94.2) | 0.1 (6.0-6.1) |
| LDTEWS:NEWS | 89.2 (89.1-89.4) | 61.1 (60.7-61.4) | 96.1 (96.1-96.1) | 7.2 (7.1-7.2) |
| >45 years old | | | | |
| iFEWS | 89.2 (89.1-89.2) | 73.3 (73.2-73.3) | 89.1 (89.1-89.1) | 7.1 (7.0-7.1) |
| LDTEWS:NEWS | 88.4 (88.3-88.4) | 68.7 (68.7-68.8) | 90.4 (90.4-90.4) | 7.5 (7.5-7.5) |
| Unplanned ICU | | | | |
| iFEWS | 88.1 (88.0-88.1) | 63.8 (63.7-63.9) | 91.2 (91.2-91.2) | 2.5 (2.5-2.5) |
| LDTEW:NEWS | 85.0 (84.9-85.0) | 57.4 (57.3-57.5) | 92.4 (92.4-92.4) | 2.6 (2.6-2.6) |
| Cardiac Arrest | | | | |
| iFEWS | 82.8 (82.7-82.9) | 57.6 (57.5-57.8) | 91.2 (91.2-91.2) | 1.2 (1.2-1.2) |
| LDTEWS:NEWS | 82.4 (82.4-82.5) | 52.3 (52.1-52.4) | 92.4 (92.3-92.4) | 1.3 (1.3-1.3) |
| Mortality | | | | |
| iFEWS | 92.4 (92.4-92.4) | 80.4 (80.3-80.5) | 89.7 (89.7-89.7) | 5.6 (5.6-5.6 |
| LDTEMS:NEWS | 91.7 (91.1-91.7) | 75.1 (75.0-75.1) | 91.1 (91.1-91.1) | 6.0 (6.0-6.0) |

For the overall dataset, iFEWS achieved a higher AUROC than LDTEWS:NEWS, [95% CI 89.5-89.5] and [95% CI 88.5-88.6] respectively. As for the 16-45 years old, iFEWS achieved an a higher AUROC [95% CI 94.2-94.3] than LDTEWS:NEWS [95% 89.1-89.2]. For the older patient group and across all outcomes, iFEWS had the highest AUROC. Thus, even on a completely independent testing set, we conclude that iFEWS had superior discriminatory performance than the multi-modal state-of-the-art EWS.

### Feature Saliency

To get a better understanding of the decision-making process of iFEWS, we examined feature saliency of the LR components. This involved investigating the weights assigned to the features after model training in the sigmoid-based layers. For example, FIG. 17 visualises the magnitude of the weights in Wₗ of the LR of the laboratory test data with sets S and U. We notice that the four additional variables considered in U are ranked within the top six weights. Additionally, UR and WBC are assigned the highest absolute weights in comparison to the other variables. This is aligned with the clinical literature where abnormal UR levels are associated with heart failure, whereas high WBC has been shown to be significantly associated with cardiovascular mortality amongst elderly patients. On the other hand, CR and POT are associated with the smallest weights.

We also examined the weights assigned to the auxiliary outputs (\hat{l}ₗ, l_{d}, and lᵥ) using the different variable types. FIG. 18 visualises the magnitude of the weights in the form of a bar chart. We observe that the highest absolute weight is assigned to the label computed using the vital sign data (lᵥ), which is approximately double the absolute weights assigned to the other variable types. We also investigated what the model learned through its embedding module 422, which converted grouped diagnosis codes into 3-dimensional vector. To do so, we first used PCA, a standard statistical procedure that are used to project the 3-dimensional vectors into a 2-dimensional space. We observe that the diagnosis groups that have a higher proportion of patients experiencing the composite outcome are clustered closer to each other.

### Clinical Utility

FIG. 19 shows the percentage of triggers, or positive alerts, produced by iFEWS in comparison to LDTEWS-NEWS at different sensitivity values (horizontal axis) in a testing set. For the 16-45 years old patients (left graph), iFEWS produces approximately 14.5% fewer positive alerts than LDTEWS:NEWS to achieve the same level of sensitivity. Across the >45 years old patients (right graph), iFEWS has approximately a 6% lower trigger rate than LDTEWS:NEWS.

The performance of iFEWS in comparison to LDTEWS:NEWS in terms of the trigger rate and the AUROC presented earlier highlights the ability of iFEWS to ease staff burden by reducing false positive alerts and providing superior discrimination ability.

1. A computer-implemented method of generating real-time alerts about a patient, comprising:
receiving vital sign data representing vital sign information obtained from the patient at one or more input times within an assessment time window;
using a Gaussian process model of at least a portion of the vital sign information to generate a time series of synthetic vital sign data based on the received vital sign data, the synthetic vital sign data comprising at least a posterior mean for each of one or more components of the vital sign information at each of a plurality of regularly spaced time points in the assessment time window;
using the generated synthetic vital sign data as input to a trained recurrent neural network to generate an early warning score, the early warning score representing a probability of an adverse event occurring during a prediction time window of predetermined length after the assessment time window; and
generating an alert about the patient dependent on the generated early warning score.

2. The method of claim 1, wherein the recurrent neural network comprises a Long Short Term Memory network.

3. The method of claim 2, wherein the Long Short Term Memory network is a bidirectional Long Short Term Memory network.

4. The method of claim 1, wherein the recurrent neural network comprises an attention mechanism.

5. The method of claim 4, wherein:
the attention mechanism computes a respective attention weight to apply to a hidden state of the recurrent neural network corresponding to each time point in the assessment time window; and
the early warning score is generated via processing of a weighted sum of the hidden states weighted by the calculated attention weights.

6. The method of claim 5, further comprising outputting an indication of a variation with time of a relevance to the generated early warning score of each of one or more components of the vital sign information based on the computed attention weights.

7. The method of claim 5, wherein the attention weights are learned, for each component of the vital sign information, based on the posterior mean of the component, at each of the time points in the assessment time window.

8. The method of claim 1, wherein:
the synthetic vital sign data comprises a posterior variance corresponding to each posterior mean;
each posterior mean corresponding to each time point is used as input to a first recurrent neural network;
each posterior variance corresponding to each time point is used as input to a second recurrent neural network; and
the early warning score is generated via processing of outputs from both the first recurrent neural network and the second recurrent neural network.

9. The method of claim 8, wherein the first recurrent neural network is a Long Short Term Memory network and the second recurrent neural network is a Long Short Term Memory network.

10. The method of claim 8, wherein:
the first recurrent neural network interacts with an attention mechanism;
the attention mechanism computes a respective attention weight to apply to a hidden state of the first recurrent neural network corresponding to each time point in the assessment time window; and
the early warning score is generated via processing of a combination of a weighted sum of the hidden states of the first recurrent neural network weighted by the computed attention weights and an output from the second recurrent neural network.

11. The method of claim 8, wherein:
the first recurrent neural network interacts with a first attention mechanism;
the second recurrent neural network interacts with a second attention mechanism;
the first attention mechanism computes a respective attention weight to apply to a hidden state of the first recurrent neural network corresponding to each time point in the assessment time window;
the second attention mechanism computes a respective attention weight to apply to a hidden state of the second recurrent neural network corresponding to each time point in the assessment time window; and
the early warning score is generated via processing of a combination of a weighted sum of the hidden states of the first recurrent neural network weighted by the computed attention weights of the first attention mechanism and a weighted sum of the hidden states of the second recurrent neural network weighted by the computed attention weights of the second attention mechanism.

12. The method of claim 1, wherein prior knowledge of either or both of the age and sex of the patient is incorporated into the mean function of the Gaussian process model.

13. The method of claim 1, wherein a radial basis function with added white noise is used as the covariance function of the Gaussian process model.

14. The method of claim 1, wherein lognormal distributions are applied as priors for the hyperparameters of the covariance function of the Gaussian process model to model a heterogeneous population of patients.

15. The method of claim 1, wherein the vital sign information comprises one or more of the following components: heart rate; respiratory rate; systolic blood pressure; diastolic blood pressure; temperature; peripheral capillary oxygen saturation; consciousness level; and whether supplemental oxygen was provided to the patient at the time of observation.

16. The method of claim 1, wherein the recurrent neural network forms part of an autoencoder and the early warning score is generated using a latent representation from the autoencoder.

17. The method of claim 16, wherein the autoencoder comprises multiple encoder channels, each encoder channel receiving vital sign data representing a different component of vital sign information.

18. The method of claim 17, wherein:
each encoder channel comprises an attention mechanism configured to compute a context vector; and
the latent representation is obtained by combining the context vectors from the multiple encoder channels and associated attention mechanisms.

19. The method of claim 18, wherein the autoencoder comprises a single decoder channel.

20. The method of claim 17, wherein parameters of the autoencoder are optimised by minimising a binary cross-entropy loss for all of the encoder channels.

21. The method of claim 1, further comprising:
receiving a diagnosis code representing a diagnosis of the patient made at a time of admission of the patient to a medical facility;
using a trained model of a relationship between diagnosis codes and probabilities of an adverse event occurring during the prediction time window to generate an early warning score based on the diagnosis code; and
obtaining a composite early warning score using a combination of at least the early warning score generated using the trained recurrent neural network and the early warning score based on the diagnosis code,
wherein the alert is generated using the composite early warning score.

22. The method of claim 1, further comprising:
receiving laboratory test data representing information obtained from one or more laboratory tests performed on the patient;
using a trained model of a relationship between laboratory test data and probabilities of an adverse event occurring during the prediction time window to generate an early warning score based on the laboratory test data; and
obtaining a composite early warning score using a combination of at least the early warning score generated using the trained recurrent neural network and the early warning score based on the laboratory test data,
wherein the alert is generated using the composite early warning score.

23. The method of claim 1, further comprising:
receiving laboratory test data representing information obtained from one or more laboratory tests performed on the patient;
receiving a diagnosis code representing a diagnosis of the patient made at a time of admission of the patient to a medical facility;
using a trained model of a relationship between laboratory test data and probabilities of an adverse event occurring during the prediction time window to generate an early warning score based on the laboratory test data;
using a trained model of a relationship between diagnosis codes and probabilities of an adverse event occurring during the prediction time window to generate an early warning score based on the diagnosis code; and
obtaining a composite early warning score using a combination of at least the early warning score generated using the trained recurrent neural network, the early warning score based on the laboratory test data, and the early warning score based on the diagnosis code,
wherein the alert is generated using the composite early warning score.

24. The method of claim 22, wherein the model of the relationship between laboratory test data and probabilities of an adverse event includes a decay term to model an effect of delay between obtaining of the laboratory test data and a time at which the composite early warning score is to be obtained.

25. A data processing apparatus comprising a processor configured to perform the method of claim 1.

26. A computer program comprising instructions which, when the program is executed by a computer, cause the computer to carry out the method of claim 1.

27. A computer-readable data carrier having stored thereon the computer program of claim 26.
